(19) United States
(12) Patent Application Publication
(10) Pub. No.: US 2010/0267002 A1
(43) Pub. Date: Oct. 21, 2010

(54) PEPTIDE HAVING LIFE-LENGTHENING EFFECT ON CELLS

(75) Inventors: Sakae TSUDA, Hokkaido (JP); Yoshiyuki Nishimiya, Hokkaido (JP); Ai Miura, Hokkaido (JP); Yu Hirano, Hokkaido (JP); Keiko Kowata, Hokkaido (JP)

Correspondence Address: SUGHRUE MION, PLLC, 2100 PENNSYLVANIA AVENUE, N.W., SUITE 800, WASHINGTON, DC, 20037, US

(73) Assignee: National Institute of Advanced Industrial Science and Technology, Tokyo (JP)

(21) Appl. No.: 12/649,083

(22) Filed: Dec. 29, 2009

(30) Foreign Application Priority Data
Apr. 20, 2009 (JP) 2009-102194

(51) Int. Cl. A01N 1/00; C07K 14/435; C12N 5/02

(52) U.S. Cl. 435/1.1; 530/324; 435/325

(57) ABSTRACT

This invention provides a substance that exhibits excellent life-lengthening effects on cells, that is excellent in terms of productivity, and that has low immunological toxicity. The present invention relates to a preservative for a biomaterial comprising (a) a peptide consisting of the amino acid sequence as shown in SEQ ID NO: 6 or (b) a peptide consisting of an amino acid sequence derived from the amino acid sequence as shown in SEQ ID NO: 6 by deletion, substitution, insertion, or addition of 1 or several amino acids and having life-lengthening effects on cells and a method for preserving a biomaterial utilizing such peptide.

### CLAIM OF PRIORITY

The present application claims priority from Japanese patent application JP 2009-102194 filed on Apr. 20, 2009, the content of which is hereby incorporated by reference into this application.

### BACKGROUND OF THE INVENTION

1. Field of the Invention

The present invention relates to a preservative for a biomaterial comprising, as an active ingredient, a peptide having life-lengthening effects on cells and a method for preserving such biomaterial using a peptide.

2. Background Art

Cells extirpated from human or animal bodies and cells grown via culture are extensively utilized in technical fields such as livestock farming or regenerative medicine, including in clinical practice. In general, such cells are handled as assemblies of several to 10,000 or more cells (i.e., cell populations or cell lines), and the survival rate thereof; i.e., the percentage of the number of viable cells relative to the total number of cells, is known to be improved by soaking the cell population in a cell preservative solution comprising inorganic salts, glycerol, sugar, amino acid, and the like. Representative examples of such cell preservative solution include a lactated Ringer's solution, the Euro-Collins solution, and the University of Wisconsin (UW) solution. With the use of such solution, however, 90% or more of the cell population dies within several hours to approximately half a day. When a cell population is cryopreserved using liquid nitrogen or a deep freezer, a majority of the cell population is exposed to physical damage that cannot be avoided during the process of freezing and thawing, and such cells disadvantageously die. Even if the number of cells that can be cultured is decreased, the number of such cells can be returned to the initial level by multiplying the cells via culture. However, many cells that are handled in actual medical practice or livestock farming cannot be cultured, or optimization of culture conditions would be very laborious and time-consuming. Thus, development of agents for lengthening cell life that allow the cell population after extirpation or culture to survive for 24 hours to several days without significantly decreasing the number of cells has been strongly desired. If such agents are developed, cryopreservation of the cell population that is to be consumed 24 hours to several days after the extirpation or culture becomes unnecessary. That is, such cell population can be preserved without damage by freezing and thawing. Also, such agents enable easy preservation of such cell population in a household refrigerator or with the use of crushed ice. Further, transportation of such cell population can be realized via a parcel delivery service or other means anywhere in the country. Specifically, extirpated cells, cultured cells, fertilized eggs, sperm, and other materials can be transported in cold storage between hospitals or between livestock farmers located in isolated locations. This can eliminate the need for hospitals or livestock framers to provide liquid nitrogen or refrigeration facilities. Thus, cost and labor that have heretofore been necessary for freezing packaging or transportation can be remarkably reduced. Such advantages can reduce energy consumption in current cold technologies, and such advantages can lead to technology upgrade in many medical services using induced pluripotent stem (iPS) cells or cells extirpated from humans.

Breeding techniques involving superovulation (hormone injections), extirpation of fertilized eggs (embryos), and preservation and transplantation of fertilized eggs (artificial insemination) in combination have been used extensively for livestock animals such as cattle, pigs, and chickens. The demand for breeding of high-grade breed varieties, such as Japanese Black Cattle (e.g., Matsusaka or Kobe beef cattle), black pigs (Berkshire pigs, Iberico pigs), and local chickens (e.g., Hinai chickens or Satsuma chickens), in particular, is rapidly increasing recent years, and 60,000 or more cattle fertilized eggs are currently distributed in Japan every year. To date, fertilized eggs of livestock animals have been frozen immediately after extirpation and transported to other livestock farmers. Thus, novel cryopreservation techniques, such as slow freezing, the Cryotop method, the Direct method, and the Open Pulled Straw method, are being developed on a daily basis, and liquid nitrogen, programmed freezers, and the like are now used in most livestock farmers. When frozen cattle eggs are used, for example, the average conception rate per egg has remained unimproved at approximately 45% over the past decade in Japan. This indicates that techniques of fertilized egg preservation have reached the technological limit in the livestock fertilized egg industry. According to conventional techniques of fertilized egg preservation, specifically, the problem of deterioration of the vital force of fertilized eggs caused by freezing or with the elapse of the time has not yet been resolved. If there were to exist a cell life-lengthening agent that could keep the fertilized eggs alive 24 hours to several days after extirpation in an unfrozen state, livestock farmers would be able to use vital fertilized eggs for breeding with the utilization of parcel delivery services or the like. Specifically, livestock fertilized eggs are cells that are to be used within 24 hours to several days after extirpation. If cell life-lengthening agents as mentioned above are developed, accordingly, it is deduced that livestock farmers or fertilized egg dealers would be relieved of the need for liquid nitrogen or expensive refrigeration facilities, such as deep freezers. More specifically, fertilized eggs could be recovered and preserved with the use of ice or household refrigerators that consume smaller amounts of energy. This could allow an increase in the number of livestock animals to a level that meets the demand for livestock animals, which is increasing every year, without increasing the amount of energy to be consumed.

Recently, possibilities in regenerative medicine have become widely recognized because of the development of “the technique for preparing induced pluripotent stem (iPS) cells” by Kyoto University. This technique comprises artificially introducing several genes into somatic cells removed from a patient to prepare totipotent cells (iPS cells or stem cells) and allowing such cells to differentiate and grow into epidermal cells, bone marrow cells, adipocytes, and the like. Transplantation of such grown cells into a patient is considered to result in regeneration of healthy cells, tissues, and organs in the patient's body. In order to implement this technique, however, cells that have been increased to the culture limit need to be kept alive without a decrease in the number thereof, and the efforts of humans (doctors) who can carry out such procedure are necessary. In the case of regenerative medicine, in particular, a cell population that has been thoroughly grown over a culture period of 1 month or longer is necessary. Many valuable cell populations that have grown to the culture limit may suffer from cryopreservation, which would disadvantageously lower the possibility of success in regenerative medicine. At present, cells extirpated from the relevant patient or someone else are used instead of cultured cells in most regenerative medical practices. If an agent that can keep the cell population alive for 24 hours to several days after culture or extirpation were to be developed, a sufficient number of cells could be used for transplantation. That is, cell populations that are kept alive in an unfrozen state for 24 hours to several days after extirpation or growth are needed in the medical field. In clinical practice, it is usual to perform surgery a few days before or after the scheduled date. If the cell population can be preserved using ice or a household refrigerator in a manner that does not require freezing and thawing and consumes a small amount of energy, some time can be saved in regenerative medical practice. It is considered that this would contribute to progress in the relevant technical field. Thus, development of a functional component that can keep the cell population alive for 24 hours to several days after extirpation or culture without significantly decreasing the number of cells and a cell life-lengthening agent comprising such component has been strongly desired.

About 20 years ago, Rubinsky et al. (University of California, U.S.A.) discovered that a biomaterial; i.e., a thermal hysteresis protein, has the function of protecting cell membranes at low temperatures of around 0° C. Such function was considered to be exhibited by a mechanism that was not available in conventional preservative solutions, the specific interaction between the thermal hysteresis protein and a cell membrane was considered to improve cell viability, and such specific interaction was considered to improve the survival ratio of fish at low temperatures. Rubinsky et al. reported in 1991 a method for improving the survival ratio of viable cells of mammalian animals comprising bringing a thermal hysteresis protein isolated and purified from a polar fish into contact with the aforementioned solution (JP Patent Publication (kohyo) No. H08-9521 B (1996)).

A thermal hysteresis protein is a biomaterial that was discovered in 1969 as a body fluid component of fish living in frigid waters. In water that is on the brink of freezing at a subzero temperature, there are numerous single ice crystals that are referred to as ice nuclei. Such ice nuclei immediately link water molecules in the vicinity thereof to grow crystals, and the grown crystals are linked to each other. Thus, ice that is usually seen is formed. Thermal hysteresis proteins accumulate on the surfaces of ice nuclei and stop the crystal growth. When a grain of ice is introduced into water of a temperature (T) maintained at around 0° C., ice is generally melted even when T is slightly raised, and such ice immediately grows when T is slightly lowered. If the temperature at which ice melts is determined to be Tₘₑₗₜ and the temperature at which ice starts to grow is determined to be T_{freeze} (i.e., the freezing point), the above general fact indicates that the correlation (Tₘₑₗₜ=T_{freeze}) is always realized with regard to common ice. When a piece of ice is introduced into an aqueous solution of thermal hysteresis proteins, however, ice melts when T is raised, although ice does not grow at all when T is lowered. This occurs because thermal hysteresis proteins are accumulated on ice surfaces and strongly act to stop the growth thereof. If T is further continuously lowered, ice starts to grow, although the temperature, i.e., the freezing point (T_{freeze}) becomes lower than Tₘₑₗₜ. In an aqueous solution of thermal hysteresis proteins, accordingly, a difference occurs between T_{freeze} and Tₘₑₗₜ. Such difference is defined as “thermal hysteresis,” and a protein that generates thermal hysteresis is defined as a “thermal hysteresis protein.” In the case of a fish thermal hysteresis protein, the thermal hysteresis value is generally 1.0° C. or higher (Kristiansen, E. and Zachariassen, 2005, Cryobiology 51, 262-280). Rubinsky et al. considered that a thermal hysteresis protein would inhibit the initiation of ice crystal growth in the blood that is almost frozen and would improve the survival ratio of fish at low temperatures.

In order to use a thermal hysteresis protein for a cell life-lengthening agent, a technique for producing a gram amount or more of such protein samples in a cost-effective manner is essential. There are 3 types of protein production techniques: 1) extraction from natural source; 2) genetic engineering; and 3) chemical synthesis. In the case of 2), however, the amounts of proteins produced from 1 liter of medium are as small as 0.1 mg to 1 mg. In the case of 3), production cost is disadvantageously very high. At present, accordingly, technique 1) (JP Patent No. 4228068) is adopted by food companies as a method for thermal hysteresis protein production, and actual production thereof is already in operation. However, thermal hysteresis protein samples extracted from natural sources contain contaminants, such as animal-derived lipids, gene fragments, protein fragments, and fever-producing substances. These substances occasionally impose strong immunological toxicity upon humans or generate unpredictable secondary damages or diseases. Thus, it is impossible to immediately use samples that are mass-produced by technique 1) in clinical practice. A technique involving introduction of cells, tissues, and organs that are soaked in preservative solutions containing fish-derived thermal hysteresis proteins into a human body presents many problems to be resolved. Thus, such technique has not yet been put to practical use.

Under such circumstances, development of components that can more effectively protect or preserve viable cells, tissues, organs, and bacteria and prolong the lives thereof has been awaited in medical fields of transplantation, and regenerative medicine, and in other fields. Thus, development of components that can be produced in amounts sufficient for practical application, that have low immunological toxicity, and that are highly safe, in addition to having a function of prolonging cell life that is superior to that of thermal hysteresis proteins, has been strongly desired.

### SUMMARY OF THE INVENTION

The present invention is intended to provide a substance that exhibits excellent life-lengthening effects on cells, good productivity, and low immunological toxicity.

### Means for Attaining the Object

The present inventors have conducted concentrated studies. As a result, they discovered that a highly safe low-molecular-weight peptide comprising 66 residues that can be mass-produced via genetic engineering exhibits strong life-lengthening effects on cells, thereby completing the present invention.

Specifically, the present invention includes the following.

(1) A preservative for a biomaterial comprising peptide (a) or (b) below:

(a) a peptide consisting of the amino acid sequence as shown in SEQ ID NO: 6; or

(b) a peptide consisting of an amino acid sequence derived from the amino acid sequence as shown in SEQ ID NO: 6 by deletion, substitution, insertion, or addition of 1 or several amino acids and having life-lengthening effects on cells.

(2) The preservative according to (1), wherein the biomaterial is a cell or a material comprising a cell.

(3) The preservative according to (2), which is used for lengthening cell life.

(4) The preservative according to any of (1) to (3), wherein the biomaterial is a cell, tissue, or organ.

(5) The preservative according to any of (1) to (3), wherein the biomaterial is a bacterium.

(6) The preservative according to any of (1) to (5), which is in the form of a liquid containing the peptide.

(7) A method for preserving a biomaterial comprising soaking a biomaterial in a liquid containing peptide (a) or (b) below:

(a) a peptide consisting of the amino acid sequence as shown in SEQ ID NO: 6; or

(b) a peptide consisting of an amino acid sequence derived from the amino acid sequence as shown in SEQ ID NO: 6 by deletion, substitution, insertion, or addition of 1 or several amino acids and having life-lengthening effects on cells.

(8) The method according to (7), wherein the biomaterial is a cell or a material comprising a cell.

The present invention provides a peptide that can preserve a cell population 24 hours to several days after extirpation or culture without significantly decreasing the number of viable cells and that has low immunological toxicity. The peptide of the present invention can be industrially produced via genetic engineering.

### BRIEF DESCRIPTION OF THE DRAWINGS

FIG. 1 shows electrophoretic patterns of high-purity samples of Nfe6 (a), Nfe8 (b), and Nfe11 (c).

FIG. 2 shows HPLC patterns of high-purity samples of Nfe6 (a), Nfe8(b), and Nfe11 (c).

FIG. 3(a) is a stereomicroscopic photograph of the HepG2 cells before the preservation experiment and FIG. 3(b) is a fluorescent microscopic photograph of the HepG2 cells stained with Calcein-AM, which is a fluorescent dye that stains only viable cells.

FIG. 4 shows microscopic photographs obtained by preserving the HepG2 cells at 4° C. for 48 hours with the use of a) an EC solution, b) an Nfe8 solution, c) an Nfe6 solution, and d) an Nfe11 solution, and staining the survived HepG2 cells yellow green (the upper photograph) and the dead HepG2 cells red (the lower photograph).

FIG. 5 shows the survival ratio of the HepG2 cells determined by preserving the cells at 4° C. for 48 hours with the use of an EC solution, an Nfe8 solution, and an Nfe11 solution based on a) the amount of WST-8, b) the amount of LDH released, and c) the amount of ATP (number of repetitions: 11).

FIG. 6 shows the survival ratio of the HepG2 cells determined by preserving the cells at 4° C. for 24 hours with the use of an EC solution, an Nfe8 solution, an Nfe6 solution, and an Nfe11 solution based on the amount of LDH released.

FIG. 7 shows the amino acid sequences of Nfe11, Nfe8, and Nfe6.

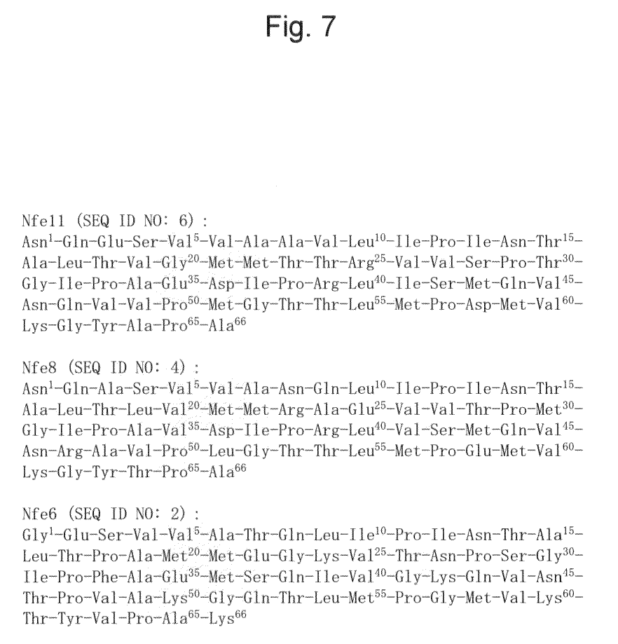

FIG. 8 shows microscopic photographs of fertilized eggs of Japanese Black Cattle preserved at 4° C. for 72 hours with the use of phosphate buffer containing Nfe11.

### DETAILED DESCRIPTION OF THE INVENTION

In the past, thermal hysteresis proteins were known to have functions of protecting biomaterials, such as cells, tissues, and organs. Thermal hysteresis proteins are also referred to as antifreeze proteins, or AFPs. Surprisingly, the present inventors discovered that there are no proportional correlations between thermal hysteresis activity and functions of protecting biomaterials and that the peptide of the present invention having no thermal hysteresis activity has excellent cell life-lengthening functions.

At first, the present inventors captured a fish (Zoarces elongatus Kner) for the purpose of searching for thermal hysteresis proteins. Thereafter, they thoroughly inspected proteins in the muscle and genes in the liver of the fish and discovered that this fish had the thermal hysteresis protein (Nfe8) consisting of the amino acid sequence as shown in SEQ ID NO: 4. Curiously enough, this fish was found to express not only Nfe8 but also as many as 12 peptides (Nfe1 to Nfe17 and Nfe9 to Nfe113) having amino acid sequence homology to Nfe8. Unexpectedly, such peptides other than Nfe8 (hereafter referred to as “Nfe peptides”) were found to have no thermal hysteresis activity (Nishimiya et al., FEBS Journal, 2005, 272, 482-492). Thereafter, the present inventors conducted concentrated studies in order to demonstrate functions of Nfe peptides having no thermal hysteresis activity.

Thermal hysteresis activity can be sufficiently assayed with the use of approximately 0.1 mg of peptides. However, a cell preservation experiment requires the use of 100 mg or more peptides. The present inventors inspected the cell life-lengthening effects of 13 types of Nfe peptides, including Nfe8. Nfe peptides with very low expression levels could not be subjected to the cell preservation experiment. The fact that the expression level is low means that it is difficult to mass-produce the Nfe peptide of interest at the industrial level. As a result of the experiment, samples of 3 types of peptides, Nfe11 (SEQ ID NO: 6), Nfe8 (SEQ ID NO: 4), and Nfe6 (SEQ ID NO: 2), were consequently obtained in amounts that could be sufficiently subjected to the experiment, and the life-lengthening effects thereof on cells were inspected. As a result, Nfe11 having no thermal hysteresis activity was found to exhibit cell life-lengthening functions that were remarkably superior to those of Nfe8 having thermal hysteresis activity. More surprisingly, another peptide exhibiting no thermal hysteresis activity (i.e., Nfe6) was found to have cell-protecting functions that were inferior to those of Nfe11.

More specifically, the present inventors discovered that a peptide consisting of the amino acid sequence as shown in SEQ ID NO: 6 (Nfe11) has cell life-lengthening functions that are stronger than those of peptides having thermal hysteresis activity; i.e., biomaterial preserving functions, despite the fact that the peptide of interest has no thermal hysteresis activity. The present inventors also discovered that, while the amount of common peptides that can be produced from 1 liter of medium is as small as 0.1 to 1 mg, the amount of Nfe11 that can be produced is one hundred to one thousand times higher than the amount of common peptides produced. The present invention has been completed based on such findings.

Accordingly, the present invention relates to a preservative for a biomaterial containing a peptide consisting of the amino acid sequence as shown in SEQ ID NO: 6. The present invention also relates to a preservative for a biomaterial containing a peptide that is functionally equivalent to the peptide consisting of the amino acid sequence as shown in SEQ ID NO: 6. The term “functionally equivalent” means that the target peptide has biological and biochemical functions equivalent to those of the peptide consisting of the amino acid sequence as shown in SEQ ID NO: 6. An example of a peptide that is functionally equivalent to the peptide consisting of the amino acid sequence as shown in SEQ ID NO: 6 is a peptide consisting of an amino acid sequence derived from the amino acid sequence as shown in SEQ ID NO: 6 by deletion, substitution, insertion, or addition of 1 or several (generally 2 to 5 and preferably 2 or 3) amino acids and having life-lengthening effects on cells. A further example thereof is a peptide consisting of an amino acid sequence having 80% or higher, preferably 90% or higher, more preferably 95% or higher, and further preferably 98% or higher identity with the amino acid sequence as shown in SEQ ID NO: 6 and having life-lengthening effects on cells. Hereafter, the peptide consisting of the amino acid sequence as shown in SEQ ID NO: 6 and peptides that are functionally equivalent thereto are referred to as “the peptide(s) of the present invention.”

In the present invention, the term “peptide” refers to a substance comprising 2 or more amino acids bound via a peptide bond (Rikagaku Eiwa Jiten (An English-Japanese Dictionary of Physical Sciences), Kenkyusha), and examples thereof include proteins, polypeptide, and oligopeptides. In the present invention, peptide salts fall within the scope of the peptide. Peptide salts are not limited, provided that they are pharmaceutically acceptable. Examples thereof include acid addition salts and base addition salts. Examples of acid addition salts include salts with inorganic acids, such as hydrochloric acid, sulfuric acid, nitric acid, and phosphoric acid, and salts with organic acids, such as acetic acid, malic acid, succinic acid, tartaric acid, and citric acid. Examples of base addition salts include salts with alkali metals, such as sodium and potassium, salts with alkaline earth metals, such as calcium and magnesium, and salts with amines, such as ammonium and triethylamine.

The peptide of the present invention can be easily produced via gene recombination techniques. When gene recombination technique is employed, DNA encoding the peptide of the present invention (e.g., DNA consisting of the nucleotide sequence as shown in SEQ ID NO: 5 or DNA equivalent thereto) is synthesized using a DNA synthesizer or the like by a conventional technique, the synthesized DNA is introduced into an adequate vector, and the resulting recombinant vector is used to transform a host such as E. coli. Subsequently, the transformant may be cultured to obtain the peptide of the present invention corresponding to the above synthesized DNA.

The term “DNA equivalent to DNA consisting of the nucleotide sequence as shown in SEQ ID NO: 5” refers to a situation in which the peptide encoded by the target DNA has biological and biochemical functions equivalent to those of a peptide encoded by DNA consisting of the nucleotide sequence as shown in SEQ ID NO: 5. An example of DNA that is functionally equivalent to DNA consisting of the nucleotide sequence as shown in SEQ ID NO: 5 is DNA that hybridizes under stringent conditions to DNA consisting of a nucleotide sequence complementary to the nucleotide sequence as shown in SEQ ID NO: 5 and encodes a peptide having life-lengthening effects on cells. Under stringent conditions, a specific hybrid is formed but a nonspecific hybrid is not formed. Examples include low stringency conditions and high stringency conditions, with high stringency conditions being preferable. Under low stringency conditions, for example, washing is carried out at 42° C. in 5×SSC and 0.1% SDS, and preferably at 50° C. in 5×SSC and 0.1% SDS, following hybridization. Under high stringency conditions, for example, washing is carried out at 65° C. in 0.1×SSC and 0.1% SDS, following hybridization. Under such stringency conditions, DNA consisting of a nucleotide sequence having high homology to the nucleotide sequence as shown in SEQ ID NO: 5 (80% or higher, preferably 90% or higher, more preferably 95% or higher, and further preferably 99% or higher homology) can hybridize to DNA consisting of a nucleotide sequence complementary to the former DNA.

The peptide of the present invention can also be obtained with the use of a cell-free peptide synthesis system (a cell-free protein synthesis system). The cell-free peptide synthesis system involves the use of a cell extract to synthesize peptides in vitro. The term “cell-free peptide synthesis system” refers to a cell-free translation system involving reading mRNA information and synthesizing peptides on the ribosome and a cell-free transcription system involving synthesis of RNA using DNA as a template. Since the cell-free peptide synthesis system can be easily modified, advantageously, an expression system that is suitable for the target peptide can be easily constructed. Details regarding the cell-free peptide synthesis system are described in, for example, JP Patent Publication (kokai) No. 2000-175695A.

Deletion, substitution, insertion, or addition of 1 or several amino acids from the amino acid sequence as shown in SEQ ID NO: 6 can be performed by a conventional technique, such as site-directed mutagenesis (Zoller et al., Nucleic Acids Res. 10, 6478-6500, 1982), by modifying the sequence of DNA encoding the peptide consisting of the amino acid sequence as shown in SEQ ID NO: 6 (e.g., DNA consisting of the nucleotide sequence as shown in SEQ ID NO: 5).

The peptide of the present invention has functions of lengthening cell life. In other words, the term “cell life-lengthening functions” refers to functions of keeping cells alive. The peptide of the present invention also has functions of protecting a cell membrane. A cell membrane has a lipid bilayer with a thickness of about 5 nm, and it allows atoms and molecules to selectively permeate the membrane; i.e., the membrane has flowability. Concentrations and conditions of substances in cells are adequately controlled by such flowability, and vital activities of cells are consequently maintained. If the lipid bilayer is damaged or the structure or flowability of the lipid bilayer is changed due to changes in external conditions, such as temperature, vital activities of cells are not maintained. Functions of protecting a cell membrane are intended to prevent the lipid bilayer from being damaged or to prevent the original structure or flowability from being lost, to thereby maintain vital activities of cells. A peptide often has some degrees of cell life-lengthening functions when it is added to a preservative solution. The duration during which such functions are exhibited is short, at approximately 24 hours, in general. In contrast, the peptide of the present invention maintains cell life-lengthening functions for 48 hours or longer, 72 hours or longer, or 96 hours or longer, and the peptide of the present invention is thus particularly excellent in this respect.

The peptide of the present invention has functions of lengthening cell life, and it thus can be used for a preservative for a biomaterial. In the present invention, biomaterials are not particularly limited, provided that they are derived from an organism. The peptide of the present invention can improve the survival ratio of any cell, regardless of cell type, provided that a cell has a lipid bilayer. Examples of biomaterials include cells and a material comprising cells, such as tissues and organs. A biomaterial may be derived from an animal or plant, with an animal-derived biomaterial being preferable. Examples of animals include mammalians (e.g., livestock animals, such as pigs, cattle, and horses, primates such as humans and monkeys, pet animals, such as dogs and cats, and rodents, such as rabbits, mice, and rats) and birds (e.g., fowls, such as turkeys and chickens).

Examples of cells to be preserved and protected include organ-derived cells, epidermal cells, pancreatic parenchymal cells, pancreatic ductal cells, renal cells, hepatic cells, blood cells, cardiac muscle cells, skeletal muscle cells, osteoblasts, skeletal myoblasts, nerve cells, vascular endothelial cells, chromocytes, smooth muscle cells, adipocytes, bone cells, cartilage cells, erythrocytes, leukocytes, eggs, sperm cells, and various types of bacteria and plant cells. Examples of tissues to be preserved and protected include epithelial tissue, connective tissue, muscle tissue, nerve tissue, dermal tissue, myeloid tissue, and corneal tissue. Examples of organs to be preserved and protected include skin, blood vessels, corneas, kidneys, the heart, the liver, the umbilical cord, bowels, nerves, lungs, placenta, the pancreas, the brain, distal portions of the extremities, and the retina. Examples of biomaterials include an organism itself, such as an embryo (a fertilized egg), the whole animal, plant seeds, and the whole plant. Specifically, the preservative for a biomaterial of the present invention can be used as, for example, a preservative for cells, a preservative for organs, a preservative for tissues, and a preservative for bacteria.

Natural protein samples extracted and purified from animals, such as fish-derived thermal hysteresis proteins, occasionally exert potent immunological toxicity on humans or generate unpredictable secondary disorders and diseases. Since the peptide of the present invention can be easily chemically synthesized or genes thereof can be easily expressed, the peptide of the present invention does not contain lipids, gene fragments, peptide fragments, or fever-producing substances that are contained in natural samples. Thus, the risk of the above-mentioned disorders or diseases or infections that may be caused with the use of animal-derived pharmaceutical products, which are well-known because of prions that are the cause of mad cow disease, is very low. The peptide of the present invention is of high safety, and it can be mass-produced in a cost-effective manner at a practical level. Also, the peptide of the present invention can be used for the long-term preservation of eggs or sperm cells, which are in high demand in the livestock and other fields, at low temperatures.

The preservative for a biomaterial of the present invention is preferably in the form of a solution containing the peptide of the present invention, more preferably a solution of the peptide of the present invention, and further preferably an aqueous solution containing the peptide of the present invention. In such a case, the concentration of the peptide of the present invention in such solution is generally 1 to 30 mg/ml, and preferably 5 to 15 mg/ml. As a result of the cell preservation experiment involving HepG2 cell lines (see the examples) via the dissolving of the peptide of the present invention (Nfe11) in the Euro-Collins solution, sufficient cell protection effects were attained at 10 mg/ml. This indicates that the existence of a given amount of peptides that is sufficient to thoroughly cover the cell membrane is preferable.

A solution used for dissolving the peptide of the present invention can be adequately selected in accordance with the application of a preservative for a biomaterial of the present invention. In general, an aqueous solution is used. Examples include various culture solutions such as Eagle's MEM, phosphate buffers such as PBS(−), Tris buffer, and physiological saline. Also, conventional organ preservative solutions, such as the Euro-Collins solution (Squifflet, J. P. et al., Transplant Proc., 13693, 1981) and the UW solution (University of Wisconsin, Wahlberg, J. A. et al., Transplantation, 43, 5-8, 1987), may be used.

The preservative for a biomaterial of the present invention may adequately comprise additives, such as an antioxidant and a stabilizer, in accordance with applications. Examples of such components include: phosphate, citrate, and other organic acids; antioxidants (e.g., SOD, vitamin E, or glutathione); low-molecular-weight polypeptide; hydrophilic polymers (e.g., polyvinyl pyrrolidone); amino acids (e.g., glycine, glutamine, asparagine, arginine, or lysine); monosaccharide, disaccharide, and polysaccharide compounds (including glucose, mannose, or dextrin); chelating agents (e.g., EDTA); sugar alcohols (e.g., mannitol or sorbitol); salt-forming counterions (e.g., sodium); nonionic surfactants (e.g., polyoxyethylene sorbitan ester (Tween (trademark)), a polyoxyethylene/polyoxypropylene block copolymer (Pluronic (trademark)), or polyethylene glycol); thrombolytic agents; vasodilators; tissue activators; catecholamine; PDEII inhibitors; calcium antagonists; β blockers; steroid drugs; fatty acid esters; antiinflammatory agents; antiallergic agents; and antihistaminic agents.

The present invention also relates to a method for preserving a biomaterial comprising soaking the biomaterial in a solution containing the peptide of the present invention. According to the method of preservation of the present invention, a biomaterial is preserved in a preservative solution at a temperature that is employed for organ preservation in clinical practice. Such temperature is generally −5° C. to 10° C., and preferably −1° C. to 4° C. By preserving a biomaterial at such low temperature, the speed at which an intracellular enzyme degrades a major cell component that is necessary for cell survival can be reduced. Preservation at low temperature does not terminate metabolism but it can delay reaction speed and cell death. Generally, a biomaterial can be preserved for 0 to 144 hours, and preferably 0 to 72 hours.

Biomaterials removed from an organism (i.e., cells, tissues, and organs) are generally soaked in a cell preservative solution and preserved in ice (around 4° C.) for the following reasons. That is, cells removed from an organism are seriously damaged by ischemic damages at a given temperature or higher. Also, cryopreservation is disadvantageous in that the survival ratio after freezing-thawing is lowered mainly because of ice recrystallization at the time of thawing, even though various freezing solutions have been developed. When cells are preserved in ice, also, cells are gradually damaged, and preservation beyond a given period of time is very difficult. In contrast, the preservative comprising the peptide of the present invention is allowed to coexist with biomaterials such as cells, tissues, and organs. Thus, the preservative of the present invention can effectively protect such materials.

Cell life-lengthening functions can be evaluated by incubating cells or a biomaterial containing cells in the presence of specimens and observing progress thereafter. A biomaterial used herein may be actually removed from an organism, or a specific cell line may be used. Evaluation of cell life-lengthening functions using a biomaterial, such as cells or tissue, removed from an organism requires the use of special facilities, techniques, and a large quantity of preservative solutions. When an established cell line is used, however, culture can be carried out with the use of relatively simple facilities, and reproducibility can be easily confirmed since the quality of such cell line is controlled on a global basis. By the evaluation method involving the use of a cell line, accordingly, various types of biomaterials, such as cells derived from various organs, can be easily tested.

When cell life-lengthening functions are evaluated with the use of cell populations or established cell lines, the percentage of the number of viable cells relative to the total number of cells; i.e., the survival ratio, can be confirmed by a method known in the art. For example, evaluation can be performed with the use of a fluorescent dye that stains viable cells (e.g., Calcein-AM) in combination with a fluorescent dye that stains dead cells (e.g., Propidium Iodide) (DeClerck et al., Journal of Immunological Methods, 172, 1994, 115; Nicoletti et al., Journal of Immunological Methods, 139, 1991, 271). With the use of the Cellstain (the Cell Double Staining Kit, Dojindo Laboratories) involving the use of Calcein-AM and propidium iodide, for example, viable cells are stained yellow green and dead cells are stained red. Thus, the cell survival ratio can be assayed.

Calcein-AM, which stains viable cells, is prepared by subjecting 4 carboxyl groups of a fluorescent molecule, Calcein (maximum absorption wavelength: 490 nm; maximum fluorescence wavelength: 515 nm), to acetoxymethyl esterification (AM). Calcein-AM does not substantially show fluorescence; however, fat solubility is elevated via acetoxymethyl esterification, Calcein-AM permeates the cell membrane, and it is then hydrolyzed by esterase in the cell. Calcein generated upon hydrolysis shows strong fluorescence and it would not permeate the cell membrane. Thus, viable cells are stained. Propidium iodide (PI, maximum absorption wavelength: 530 nm; maximum fluorescence wavelength: 620 nm), which stains dead cells, binds to a nucleic acid and emits fluorescence. Since PI does not permeate the cell membrane, PI is taken up by a cell having a significantly damaged cell membrane, and a cell nucleus is thus stained.

Cell injury, i.e., cell membrane defect, may be quantified to measure the cellular survival ratio (the document: T. Decker and M. L. L. Matthes, Journal of Immunological Methods, 115, 1988, 61). For example, a method involving the use of an amount of lactate dehydrogenase (LDH) as an indicator is well known. LDH is a cytoplasmic enzyme that stably exists in every cell, and it is immediately released into a culture supernatant when the cell membrane is damaged. As a result of the reaction among the substrate contained in the kit, a catalyst (diaphorase), and LDH released into the cell supernatant, red formazan that has a peak at the absorption wavelength of about 500 nm is generated from a tetrazolium salt, INT, (2-[4-indophenyl]-3-[4-nitrophenyl]-5-phenyl tetrazolium chloride). An increase in the number of dead cells or cells having damaged cell membranes is observed as an increase in LDH enzyme activity in the culture supernatant. An increase in LDH enzyme activity in the culture supernatant is linearly correlated with the amount of red formazan generated within a given period of time. Thus, quantification of stable LDH enzyme enables estimation of the number of dead cells resulting from broken cell membranes. Also, a tetrazolium salt, WST-8, (2-(2-methoxy-4-nitrophenyl)-3-(4-nitrophenyl)-5-(2,4-disulfophenyl)-2H-tetrazolium monosodium salt) is reduced by dehydrogenase present in a living cell to generate highly sensitive and water-soluble formazan having absorption at 450 nm. Measurement of the amount of formazan also enables accurate quantification of viable cells.

Hereafter, examples of the present invention are described, although the present invention is not limited to the examples.

### EXAMPLES

### (1) Construction of Expression Vector that Expresses Nfe6, Nfe8, and Nfe11

A fresh fish, Zoarces elongatus Kner, was caught off of the eastern coast of Hokkaido, mRNA was extracted and purified from its liver, and a cDNA library was then prepared using the ZAP-cDNA Synthesis Kit (TOYOBO). PCR was carried out using the cDNA library as a template and oligonucleotides having the nucleotide sequences as shown in SEQ ID NOs: 7 and 8 as primers, and 500 to 700-bp DNA fragments were cloned into pGEM-T Easy (Promega). By decoding the DNA sequences, cloning of DNA fragments comprising the sequences of Nfe6, Nfe8, and Nfe11 shown in SEQ ID NOs: 1, 3, and 5, respectively, was confirmed. Since DNA of the secretory signal sequence was observed at a site upstream of DNA encoding Nfe6, Nfe8, and Nfe11, a DNA fragment comprising the Nfe6 sequence was subjected to PCR using the oligonucleotides represented by the nucleotide sequences as shown in SEQ ID NOs: 9 and 8 as a primer pair, a DNA fragment comprising the Nfe8 sequence was subjected to PCR using the oligonucleotides represented by the nucleotide sequences as shown in SEQ ID NOs: 10 and 8 as a primer pair, and a DNA fragment comprising the Nfe11 sequence was subjected to PCR using the oligonucleotides represented by the nucleotide sequences as shown in SEQ ID NOs: 11 and 8 as a primer pair, to remove secretory signal sequences. The NdeI sites have been introduced into primers comprising the nucleotide sequences as shown in SEQ ID NOs: 9 to 11, and the XhoI site has been introduced into a primer comprising the nucleotide sequence as shown in SEQ ID NO: 8. After the DNA fragments obtained via PCR and pET20b (Novagen) were digested with NdeI and XhoI, the ligation reaction was carried out to obtain pET20NFE6, pET20NFE8, and pET20NFE11 plasmids.

### (2) Expression of Nfe6, Nfe8, and Nfe11 Gene Recombinants

BL21 (DE3) (Novagen) transformed with pET20NFE6, pET20NFE8, or pET20NFE11 was subjected to agitation culture at 28° C. for 24 hours in 2×YT medium containing ampicillin at 100 μg/ml. A culture solution was transferred to a fresh 2×YT medium containing ampicillin at 100 μg/ml in an amount of 1/100 of the medium by volume, and agitation culture was conducted at 28° C. IPTG (isopropyl-β-D(−)-thiogalactopyranoside) was added to a final concentration of 0.5 mM at O.D.₆₀₀=0.5 to induce expression, and culture was conducted for an additional 18 hours.

### (3) Purification of Nfe6, Nfe8, and Nfe11 Gene Recombinants

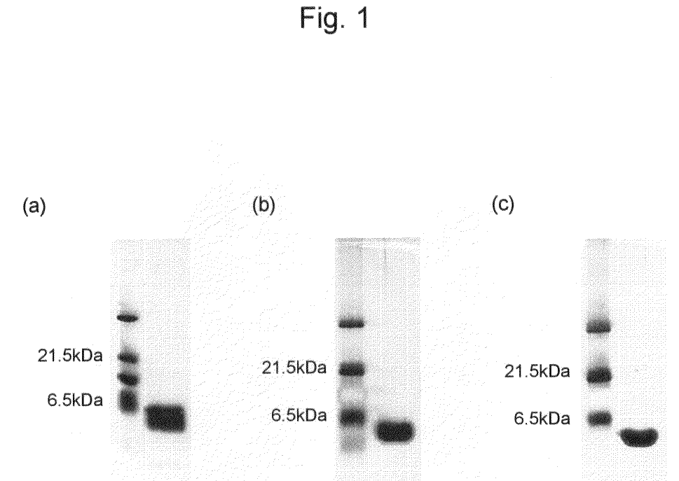

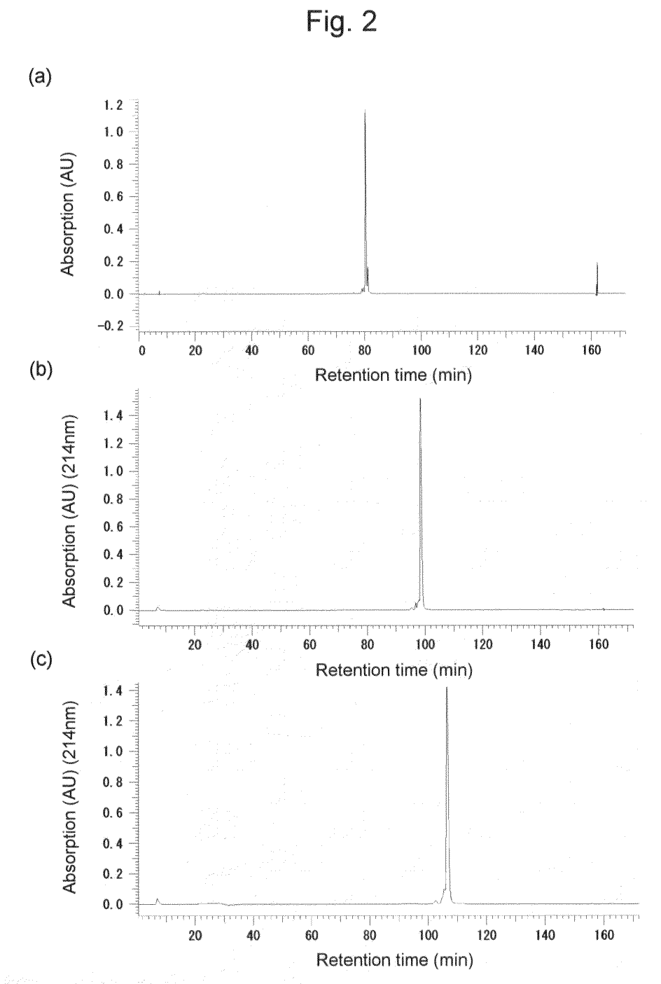

Nfe6, Nfe8, and Nfe11 gene recombinants were purified in the manner described below. The culture solution was centrifuged at 5,000 rpm for 15 minutes at 4° C. and cells were recovered. TE buffer (10 mM Tris-HCl/1 mM EDTA; pH 8.0) in an amount of 1/20 of the culture solution and phenylmethylsulfonyl fluoride (PMSF) were added thereto to a concentration of 0.1 mM, and cells were suspended. The suspension was subjected to freezing once and then to thawing. Thereafter, the cells were ground using an ultrasonic apparatus. The solution of ground cells was centrifuged at 11,000 rpm for 20 minutes at 4° C., and the supernatant was recovered. Citric acid monohydrate (13.2 g/l) and sodium chloride (29.2 g/l) were dissolved in the supernatant, and the resulting solution was allowed to stand at 4° C. for 1 hour. The resulting precipitate was centrifuged at 6,000 rpm at 4° C. for 30 minutes and a supernatant containing a target protein was recovered. The supernatant was allowed to pass through the Sephadex G-25 gel filtration column (5.0 cm (I.D.)×26 cm (height); volume: 500 ml, GE Healthcare) using a 5 mM sodium citrate solution as a mobile phase, and a protein solution, which had been replaced with a 5 mM sodium citrate solution, was thus obtained. Subsequently, the pH level of the protein solution was adjusted to 2.9 with a citric acid solution, and the resultant was allowed to stand at 4° C. overnight to subject contaminating proteins to acid denaturation and precipitation. The resulting precipitate was removed via centrifugation at 6,000 rpm at 4° C. for 30 minutes to obtain a supernatant containing the target protein. The cation exchange High-S column (I.D.: 5.0 cm; height: 4.6 cm; volume: 90 ml, Bio-Rad) was equilibrated with 50 mM sodium citrate buffer (pH 2.9), and the recovered supernatant was then allowed to pass through the column. The target protein that had adsorbed to resin was eluted with 50 mM sodium citrate/330 mM NaCl (pH 2.9) and recovered. FIG. 1(a) to FIG. 1(c) show the results of SDS-PAGE of the protein solution fractionated with the use of a cation exchange column. A band was detected at around 4.5 kDa for Nfe6, for Nfe8, and for Nfe11. Type III thermal hysteresis proteins, (antifreeze proteins), to which Nfe8 corresponds, are known to exhibit an electrophoresis band at around 4.5 kDa, which is smaller than actual molecular weight, as a result of SDS-PAGE (Hew et al., 1984, J. Comp. Physiol. B155, 81-88). Accordingly, the results of electrophoresis demonstrate that Nfe6, Nfe8, and Nfe11 are highly purified. An aqueous solution of the purified products was dialyzed 5 times against a solution of 20 mM ammonium acetate in an amount 10 times higher than that of the aqueous solution, and the resultant was then subjected to lyophilization to obtain sample powders. Some of such powders were dissolved in ultrapure water and the solution was subjected to HPLC analysis using ODS-80TS (TOSOH). The results are shown in FIG. 2(a) to FIG. 2(c). The results indicate that peak areas of Nfe6, Nfe8, and Nfe11 are 90% or greater than the entire areas and peptide purity is 90% or higher. The average yields were about 0.1 g (Nfe6), 18 mg (Nfe8), and 0.1 g (Nfe11) per liter of medium.

### (4) Preparation of Preservative Solutions

The solutions A to D shown below were prepared and used for the cell preservation experiment.

Solution A1): A solution (the Euro-Collins solution or the EC solution) prepared by adding a 50% glucose solution (Daiichi Sankyo Co., Ltd.) to a commercially available Euro-Collins solution (a preservative solution for isolated kidney, Prom, From Co., Ltd.) at 35 ml/l.

Solution A2): A solution prepared by adding Triton-X to the stock solution of a commercially available Euro-Collins solution at 10 ml/l.

Solution B): A solution of Nfe8 powders dissolved in A1) at 10 mg/ml (the Nfe8 solution).

Solution C): A solution of Nfe6 powders dissolved in A1) at 10 mg/ml (the Nfe6 solution).

Solution D): A solution of Nef11 powders dissolved in A1) at 10 mg/ml (the Nfe11 solution).

Here, Nfe8 has thermal hysteresis activity, although Nfe6 and Nfe11 do not have any thermal hysteresis activity.

### (5) Preparation of Cells

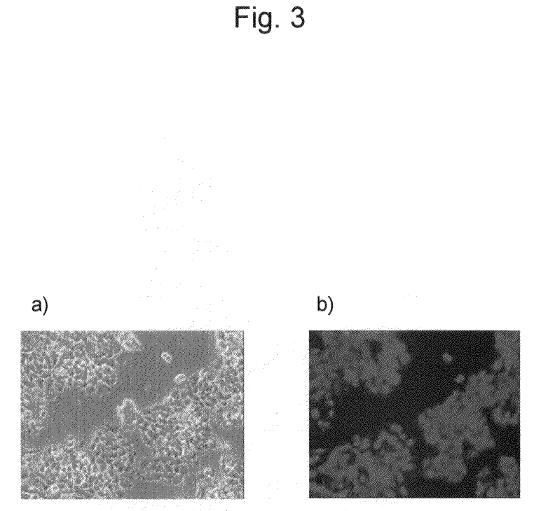

Nfe6-, Nfe8-, and Nfe11-based confirmation of cell protecting effects and cell preserving effects was performed with the use of the HepG2 cell line (FIG. 3) derived from the human liver (purchased from Riken Cell Bank). The experiment was carried out using the HepG2 cells that had been cultured for about a week in Dulbecco's Modified Eagle's Medium (DMEM) (Sigma-Aldrich, Japan) (a culture solution) containing 10% bovine serum (FBS, Gibco) and 50 μg/ml penicillin/streptomycin (Invitrogen). FBS was inactivated by heating at 56° C. for 30 minutes and then used. Culture was conducted in a CO₂ incubator (SANYO) using a flask in an atmosphere of 5% CO₂ at 37° C. The HepG2 cells that had reached subconfluent conditions (i.e., conditions immediately before the container becomes filled with cells) were treated with a trypsin solution (Wako Pure Chemical Industries, Ltd.) to prepare a cell suspension. The cell suspension was sowed on a cell culture microplate, culture was conducted in a CO₂ incubator (5% CO₂, 37° C.) overnight, and the resultant was then used for the preservation experiment. In all the experiments described below, cells were preserved at 4° C.

### (6) Evaluation of Cell Survival Ratio

The cells that had been subjected to preservation treatment for 48 hours were stained with Cellstain (Cell Double Staining Kit, available from Dojindo Laboratories), and the cells were observed under a fluorescent microscope (Olympus IX70) to evaluate the survival ratio. Cellstain (Cell Double Staining Kit) involves the use of Calcein-AM fluorescent dye, which stains viable cells, in combination with PI (Propidium iodide) fluorescent dye, which stains dead cells. This kit stains viable cells yellow green and dead cells red. The preservation experiment was carried out using a 4-well cell culture plate (Nalge Nunc International K.K.) and the HepG2 cells prepared by the method described in (5). The prepared cells were washed, the medium was replaced with the preservative solution described in (4), and the resultants were preserved at 4° C. for 24 hours and 48 hours. After the preservation, the cells were transferred to the incubator at 37° C., the cells were allowed to stand for 1 hour, the double staining solutions were added to final concentrations of 2 μM of Calcein-AM and 4 μM of PI, and the resultants were incubated at 37° C. for 15 minutes. Thereafter, the cell culture plate was mounted on a fluorescent microscope, and “viable cells stained yellow green” were observed using a fluorescence cube for GFP (excitation filter: BP460-480; dichroic mirror: DM450; absorption filter: BA460-510). Also, “dead cells stained red” were observed using a fluorescence cube for YFP (excitation filter: BP490-500; dichroic mirror: DM505; absorption filter: BA515-560).

The cell survival ratio after 24-hour preservation was also estimated using the WST-8-based Cell Counting Kit-8 (Dojindo Laboratories). The preservation experiment was carried out using a 4-well cell culture plate (Nalge Nunc International K.K.) and the HepG2 cells prepared by the method described in (5). The prepared cells were washed, the medium was replaced with the preservative solution described in (4), and the resultants were preserved at 4° C. for 24 hours and 48 hours. The preservative solution was replaced with DMEM medium, culture was conducted in a CO₂ incubator (5% CO₂, 37° C.) for 2 hours, WST-8 was added, and culture was conducted for an additional 2 hours. The amount of water-soluble formazan in the medium was evaluated by measuring the absorbance at 450 nm using a microplate reader (Sunrise Remote, Wako Pure Chemical Industries, Ltd.), and the percentage of viable cells after the cell preservation experiment was determined using the number of cells determined by adding WST-8 to the HepG2 cells before the preservation experiment as a control (100%).

Further, the cell survival ratio after 24-hour-preservation was estimated using the LDH Cytotoxicity Detection Kit (Takara Bio Inc.). The LDH amount was measured by sowing the cell suspension prepared by the method described in (5) on a 4-well cell culture plate (Nalge Nunc International K.K.) and subjecting the resultants to the preservation experiment at 4° C. for 24 hours and for 48 hours. The supernatant on the plate after preservation was transferred to a fresh 96-well plate, the reagent was added, and the resultant was then incubated at 25° C. for 30 minutes. The amount of red formazan generated was evaluated by assaying the absorbance at 490 nm using a microplate reader (Sunrise Remote, Wako Pure Chemical Industries, Ltd.). In this experiment, the LDH amount when the HepG2 cells had been preserved for 24 hours using a cell preservative solution containing 1% Triton X (the A2 solution) was designated as 100% and the percentage of LDH in the experiment was determined based thereon.

Since the metabolism is reduced at low temperatures, substantially no ATP is synthesized in cells. When cells are damaged, intracellular ATP is consumed in order to maintain the homeostatic conditions. Thus, measurement of the intracellular ATP concentration after low-temperature-preservation enables evaluation of low-temperature stress imposed on cells. The ATP concentration was measured using a 96-well cell culture plate for emission measurement (Nalge Nunc International K.K.) and the HepG2 cells prepared by the method described in (5). The prepared cells were washed, the medium was replaced with the preservative solution described in (4), and the resultants were preserved at 4° C. for 24 hours and 48 hours. ATP concentration after preservation was evaluated by adding a luminescent reagent (CellTiter-Glo, Promega) to wells and assaying the luminescent intensity using a microplate reader (Wallac 1420 ARVOsx, PerkinElmer Life Sciences, Japan). In this experiment, the medium used for the cells before preservation was replaced with the Euro-Collins solution (the A1 solution), and the ATP concentration at this time was designated as 100% to determine the ATP concentration (%) in each experiment.

### (7) Results of Observation of HepG2 Cells after 48-Hour Preservation Using Fluorescent Dye

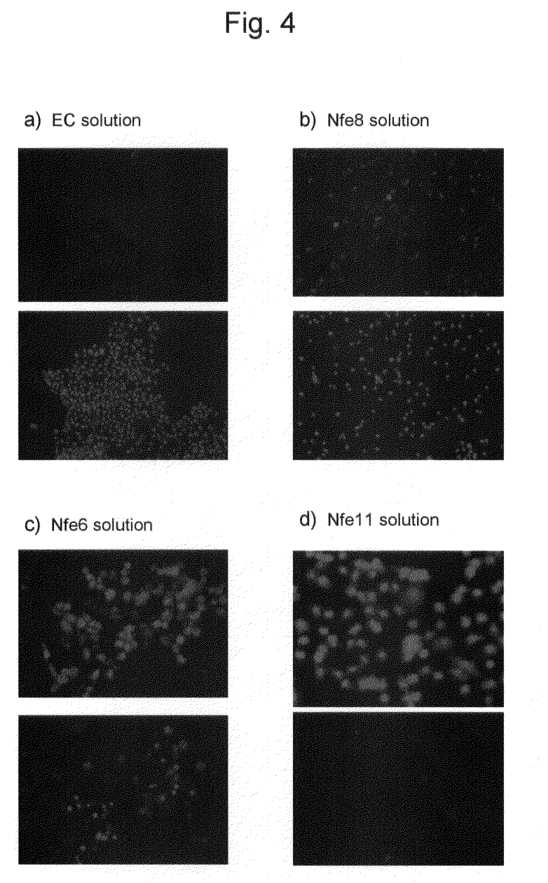

The HepG2 cell preservation experiment was carried out for 48 hours using the Euro-Collins solution, the Nfe8 solution, the Nfe6 solution, and the Nfe11 solution, and the obtained fluorescent microscopic images are shown in FIG. 4(a) to FIG. 4(d). The cells stained green (the upper portion of FIG. 4) represent viable cells after the preservation experiment, and the cells stained red (the lower portion of FIG. 4) represent dead cells after the preservation experiment. When the Euro-Collins solution (the EC solution) was used, it was found that the cell population had completely died (FIG. 4a). As a result of another experiment, it was found that 85% or more of the HepG2 cells had died 24 hours after the preservation experiment. When the Nfe8 solution was used, the cell survival ratio was as low as about 25% (FIG. 4b). When the Nfe6 solution was used, the cell survival ratio was also as low as about 40% (FIG. 4c). When the Nfe11 solution was used, however, the cell survival ratio was as high as about 75% (FIG. 4b). The results of experiment demonstrate that Nfe11 having no thermal hysteresis activity has cell protecting functions and cell life-lengthening functions that are much better than those of Nfe8 having thermal hysteresis activity. Also, cell protecting functions were found to significantly differ between Nfe11 and Nfe6, which do not have thermal hysteresis activity. Thus, it was concluded that Nfe11 has high specificity regarding cell protecting functions and cell life-lengthening functions.

### (8) Results of Assay of Survival Ratio of HepG2 Cells (1)

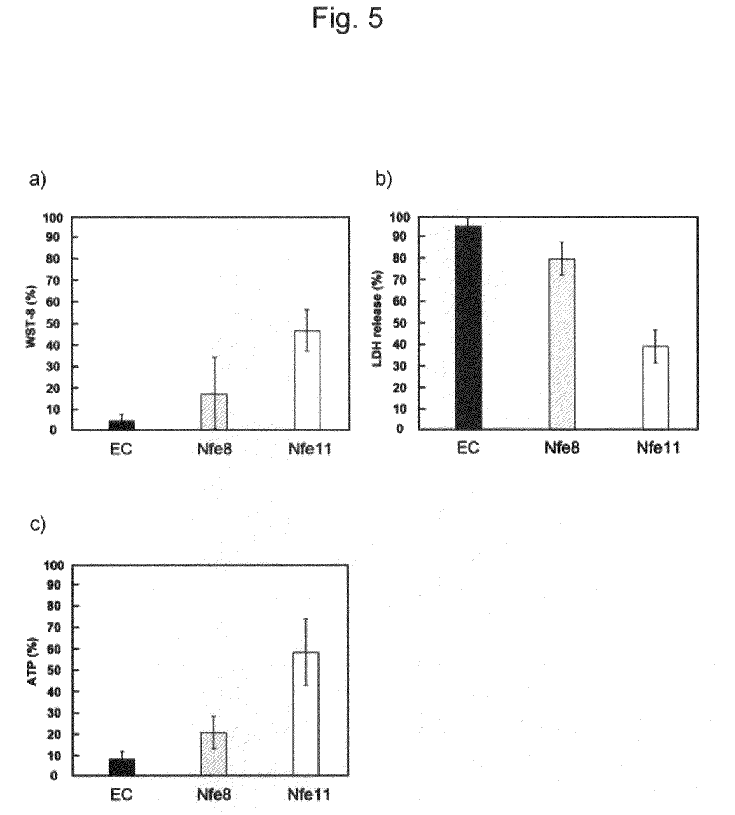

FIG. 5 shows the results of quantification of the survival ratio of the HepG2 cells after preservation thereof using the EC solution, the Nfe8 solution, and the Nfe11 solution described in (4) at 4° C. for 48 hours. The results attained by repeating the experiment 11 times in total are summarized. By the method for measuring the amount of WST-8; i.e., the dehydrogenase capacity of viable cells, as shown in FIG. 5, the survival ratios of the HepG2 cells when using the EC solution, the Nfe8 solution, and the Nfe11 solution were estimated to be about 4%, 16%, and 48%, respectively (FIG. 5a). As a result of measurement of the amount of LDH released, the cell survival ratios when the EC solution, the Nfe8 solution, and the Nfe11 solution were used were estimated to be about 4%, 19%, and 62%, respectively (FIG. 5b). Since the amount of LDH released indicates the quantity of cells with broken membranes, the value obtained by subtracting the value shown in the chart in FIG. 5b from 100 was determined to be the cell survival ratio. By the method for assaying the amount of ATP, i.e., the amount of energy of cells, the cell survival ratios when the EC solution, the Nfe8 solution, and the Nfe11 solution were used were estimated to be 6%, 20%, and 60%, respectively. The results of experiment demonstrate that Nfe11 having no thermal hysteresis activity has cell protecting functions and cell life-lengthening functions that are better than those of Nfe8 having thermal hysteresis activity.

### (9) Results of Assay of Survival Ratio of HepG2 Cells (2)

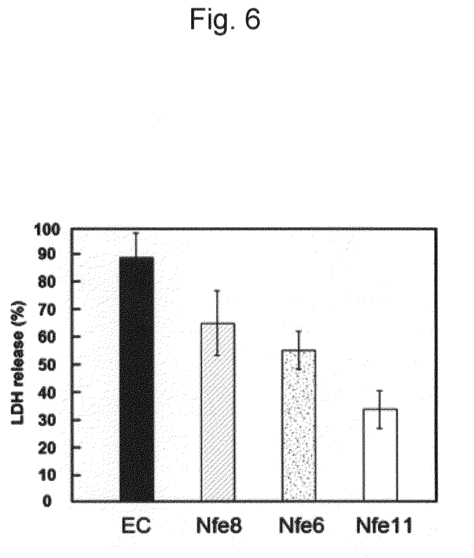

FIG. 6 shows the results of quantification of the survival ratio of the HepG2 cells after preservation thereof using the EC solution, the Nfe8 solution, the Nfe6 solution, and the Nfe11 solution described in (4) at 4° C. for 24 hours. The results attained by repeating the experiment 11 times in total are summarized. As shown in FIG. 6, the survival ratios when using the EC solution, the Nfe8 solution, the Nfe6 solution, and the Nfe11 solution were estimated to be about 89%, 67%, 56%, and 34%, respectively, based on the assay of the amount of LDH (FIG. 5). The results of experiment demonstrate that Nfe11 having no thermal hysteresis activity has cell protecting functions and cell life-lengthening functions that are much better than those of Nfe8, which is a thermal hysteresis protein. Also, cell life-lengthening functions of Nfe11 and Nfe6, which do not have thermal hysteresis activity, were found to differ significantly. Thus, it was concluded that Nfe11 has high specificity regarding cell protecting functions and cell life-lengthening functions.

### (10) Assay of Survival Ratio of Fertilized Eggs of Japanese Black Cattle

The fertilized eggs of Japanese Black Cattle were subjected to the preservation experiment at 4° C. for 72 to 96 hours (3 to 4 days) using Nfe11, and the survival ratio of the fertilized eggs after preservation was assayed. At the outset, the follicle-stimulating hormones (FSH) were used to subject superovulated male cattle to artificial insemination. Seven days thereafter, 3 to 6 fertilized eggs at the initial blastocyst stage were removed from the salpinx of the male cattle and introduced into a straw having a diameter of 400 micrometers. In this case, Nfe11 powder samples were dissolved in a phosphate buffer (PBS) to a concentration of 5 mg/ml or 10 mg/ml, and the recovered fertilized eggs were soaked in the resulting solutions. Both ends of the straw were sealed, the resultant was allowed to stand in a refrigerator at 4° C., and the preservation experiment was carried out for 72 hours to 96 hours. Survival of the fertilized eggs after the preservation experiment was evaluated using a microscopic observation. As a result, the data as shown below were obtained.

1) As a result of the preservation experiment of cattle fertilized eggs using 10 mg/ml AFP at 4° C. for 96 hours, the survival ratio was found to be 50%.

2) As a result of the preservation experiment of cattle fertilized eggs using 5 mg/ml AFP at 4° C. for 96 hours, the survival ratio was found to be 50%.

3) As a result of the preservation experiment of cattle fertilized eggs using 5 mg/ml AFP at 4° C. for 72 hours, the survival ratio was found to be 67%.

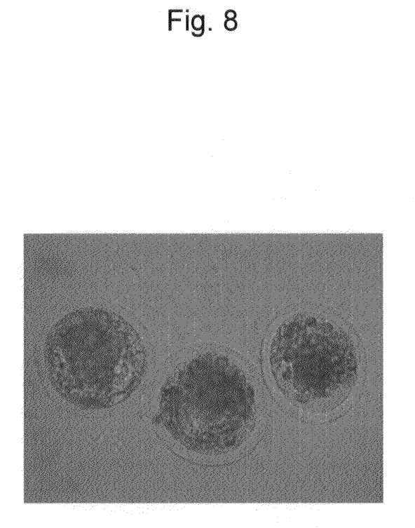

FIG. 8 shows an example of a microscopic photograph of cattle fertilized eggs after the 72-hour preservation experiment. This photograph indicates that 2 cattle fertilized eggs (shown on the left) among the 3 cattle fertilized eggs maintain very good conditions after the 72-hour preservation experiment at low temperature. Thus, Nfe11 was found to exhibit excellent life-lengthening effects on cattle fertilized eggs.

All publications, patents, and patent applications cited herein are incorporated herein by reference in their entirety.

### INDUSTRIAL APPLICABILITY

Since the peptide of the present invention can be mass-produced by general genetic engineering techniques, the peptide of the present invention can be easily obtained without being affected by conditions, such as natural sources (e.g., fish) or weather. Thus, the present invention can remarkably contribute to promotion of utilization of cell preservation at low temperature and promotion of fundamental and applied research in all biotechnological fields, which are fundamental to such preservation technique. About 0.1 g of the peptide of the present invention can be produced from 1 liter of medium. Thus, the present invention is very useful on a practical level.

1. A preservative for a biomaterial comprising peptide (a) or (b) below:
(a) a peptide consisting of the amino acid sequence as shown in SEQ ID NO: 6; or
(b) a peptide consisting of an amino acid sequence derived from the amino acid sequence as shown in SEQ ID NO: 6 by deletion, substitution, insertion, or addition of 1 or several amino acids and having life-lengthening effects on cells.

2. The preservative according to claim 1, wherein the biomaterial is a cell or a material comprising a cell.

3. The preservative according to claim 2, which is used for lengthening cell life.

4. The preservative according to claim 1, wherein the biomaterial is a cell, tissue, or organ.

5. The preservative according to claim 1, wherein the biomaterial is a bacterium.

6. The preservative according to claim 1, which is in the form of a liquid containing the peptide.

7. A method for preserving a biomaterial comprising soaking a biomaterial in a liquid containing peptide (a) or (b) below:
(a) a peptide consisting of the amino acid sequence as shown in SEQ ID NO: 6; or
(b) a peptide consisting of an amino acid sequence derived from the amino acid sequence as shown in SEQ ID NO: 6 by deletion, substitution, insertion, or addition of 1 or several amino acids and having life-lengthening effects on cells.

8. The method according to claim 7, wherein the biomaterial is a cell or a material comprising a cell.

9. The method according to claim 7, wherein the biomaterial is a cell, tissue, or organ.

10. The method according to claim 7, wherein the biomaterial is a bacterium.


SEQUENCE LISTING (11 sequences)

SEQ ID NO: 1 - DNA, 511 bp, Zoarces elongatus Kner
  CDS (59)..(322)
gctttcacttcgttctccgataattaattaattaattaattaagtctcagccacagccatgaagtccgttattttaaccggtttgcttttcgtcctcctttgtgtcgaccacatgagttcagccggcgagtccgtggtggccacccagctgatccccataaatactgccctgactccggcgatgatggaggggaaggtgaccaacccaagcggcatcccgttcgcggagatgtcccaaatagtggggaagcaagtgaacacgccagtggctaagggccaaaccctcatgccaggcatggtgaaaacgtacgtcccggcaaagtagttccgagggtgccaaggagcttcttcccaaaaccaaaagaagaaatgccccctctcacaattaaccttgtttttgtcacaaacccaagtctgtccggatgttaactgaacatgtcaaaacctgtggagactgttgagatttgatgttctgaaaagataaagcctataaataaaatgtgcccaaaaa

SEQ ID NO: 2 - protein, 66 aa, Zoarces elongatus Kner
GESVVATQLIPINTALTPAMMEGKVTNPSGIPFAEMSQIVGKQVNTPVAKGQTLMPGMVKTYVPAK

SEQ ID NO: 3 - DNA, 483 bp, Zoarces elongatus Kner
  CDS (22)..(285)
gattaagtctcagccacagtcatgaagtcagtcattttaactggtttgcttttcgtcctcctttgtgtcgaccacatgagttcagccaaccaggcgtccgtggtggccaaccagctgatccccataaatactgccctgactctggtgatgatgagggcggaggtggtcaccccaatgggcatccccgccgtggacattccccgattagtctcaatgcaagtgaacagggcagtgccgttgggcacaaccctcatgccagagatggtgaaagggtacaccccggcttagtagttctgagggtgccaaggagtttcttcccaaagccaaaagaagaaatgcctcctctcacaactaaccttgttttgtcacaaacccaagtctgtccgtatgttaactgaacatgtcaaaacctgtgaagactatgttgagatttgatgttctgaaaagataaagcatttaaataaaatgtgcccaaaaaaaaaa

SEQ ID NO: 4 - protein, 66 aa, Zoarces elongatus Kner
NQASVVANQLIPINTALTLVMMRAEVVTPMGIPAVDIPRLVSMQVNRAVPLGTTLMPEMVKGYTPA

SEQ ID NO: 5 - DNA, 501 bp, Zoarces elongatus Kner
  CDS (62)..(325)
gctttcacttcgttctccgctaattaattaataactaattaattaagtctcagccacagccatgaagtcagtcattttaactggtttgttcttcgtcctcctttgtgtcgaccacatgagttcagccaaccaggagtccgtggtggccgccgttctgatccccataaatactgccctgactgtggggatgatgacgacacgggtggtctccccaacgggcatccccgccgaggacattccccgattaatctcaatgcaagtgaaccaggtagtgccgatgggcacaaccctcatgccagacatggtgaaagggtacgccccggcttagtagttctgagggtgcccaggagtttcttcccaaagccaaaagaagaaatgcctcctctcacaattaaccttgtttttgtcacaaacccaagtctgtccggatgttaactgaacatgtcaaaacctttggagactatgttgagatttgatgttctgaaaagataaagcatttaa

SEQ ID NO: 6 - protein, 66 aa, Zoarces elongatus Kner
NQESVVAAVLIPINTALTVGMMTTRVVSPTGIPAEDIPRLISMQVNQVVPMGTTLMPDMVKGYAPA

SEQ ID NO: 7 - DNA, 19 bp, Artificial Sequence
  Synthetic oligonucleotide
tcgggaattcggcacgagg

SEQ ID NO: 8 - DNA, 21 bp, Artificial Sequence
  Synthetic oligonucleotide
gagagaactagtctcgagttt

SEQ ID NO: 9 - DNA, 31 bp, Artificial Sequence
  Synthetic oligonucleotide
ggaattccatatgggcgagtccgtggtggcc

SEQ ID NO: 10 - DNA, 34 bp, Artificial Sequence
  Synthetic oligonucleotide
gggatatccatatgaaccaggcgtccgtggtggc

SEQ ID NO: 11 - DNA, 34 bp, Artificial Sequence
  Synthetic oligonucleotide
gggatatccatatgaaccaggagtccgtggtggc